(19) United States
(12) Plant Patent Application Publication
(10) Pub. No.: US 2014/0283255 P1
(43) Pub. Date: Sep. 18, 2014

(54) Perovskia plant named 'WALPPB'

(50) ;

(71) Applicant: Kevin J. Walters, Zeeland, MI (US)

(72) Inventor: Kevin J. Walters, Zeeland, MI (US)

(21) Appl. No.: 13/815,844

(22) Filed: Mar. 16, 2013

(51) Int. Cl. A01H 5/00

(52) U.S. Cl. PLT/263.1

(57) ABSTRACT

The new and distinct cultivar of ornamental hardy Russian Sage, Perovskia ‘WALPPB’ with finely dissected silvery foliage, erect branching spikes producing a short, compact habit with numerous flowers over a long period.

### BOTANICAL CLASSIFICATION

Perovskia atriplicifolia (Benth.)

### VARIETY DENOMINATION

‘WALPPB’

### BACKGROUND OF THE INVENTION

The present invention relates to a new and distinct plant of Russian Sage, botanically known as Perovskia atriplicifolia ‘WALPPB’ and will be referred to hereinafter by its cultivar name ‘WALPPB’ and the new plant. The new plant cultivar of Perovskia is a hardy herbaceous perennial grown for landscape use.

Perovskia ‘WALPPB’ is from the selection of seed from Perovskia atriplicifolia ‘Little Spire’ collected by the inventor Kevin J. Walters in the summer of 2006 at a nursery in Zeeland, Mich. All subsequent evaluations were performed in a full-sun trial garden at the same nursery with loam sand soil with irrigation and fertilizer as required. The single seedling plant was selected for further evaluation during the summer of 2008 and given the breeder designation “06-short.” The plant was propagated by stem cuttings at the same nursery in Zeeland, Mich. and the original plant and the cuttings evaluated over the next three years until approved for introduction in the summer of 2010. The resultant asexually propagated cuttings have been stable and identical to the original plant in successive asexual propagations.

The new cultivar Perovskia ‘WALPPB’ is sold under the trademark PEEK-A-BLUE.

### SUMMARY OF THE INVENTION

Perovskia atriplicifolia ‘WALPPB’ differs from its parent as well as all other hardy Russian Sage known to the applicant in a combination of traits. The following characteristics in combination distinguish ‘WALPPB’ from all other Perovskia plants known to the inventor:

1. Erect and compact habit

2. Long flowering season

3. Lavender blue flowers

4. Silvery green finely dissected foliage

The nearest comparison plants are Perovskia ‘Filagran’ (not patented) ‘Blue Spire’ (not patented) and the parent ‘Little Spire’ U.S. Plant Pat. No. 11,643. The new plant compares to ‘Filagran’ and ‘Blue Spires’ in being about 20% shorter, longer flowering as well as having finer dissected foliage than the latter. ‘WALPPB’ has finer dissected foliage than ‘Little Spire’ and is about 20% shorter. The secondary branches of ‘WALPPB’ are at about 45 degrees above horizontal whereas the secondary branches of ‘Little Spire’ are closer to 55 to 60 degrees above horizontal. The hairs on the calyxes of ‘WALPPB’ are a dusty lighter violet compared to the medium violet hair on the calyxes of ‘Little Spire’.

### BRIEF DESCRIPTION OF THE DRAWINGS

The photograph of the new plant demonstrate the overall appearance of the plant, including the unique traits. The colors are as accurate as reasonably possible with color reproductions. Ambient light spectrum, source and direction may cause the appearance of minor variation in color. The plant used in the photograph is a two-year old plant grown in full sun field in loamy sand soil in trial gardens of a nursery in Zeeland, Mich.

FIG. 1 shows a two-year old Perovskia atripicifolia ‘WALPPB’ in the landscape.

### DETAILED BOTANICAL DESCRIPTION

The following is a detailed description of Perovskia atripicifolia ‘WALPPB’ as observed for five years in trial gardens and production fields at a nursery in Zeeland, Mich. The following description is of a two-year old plant of Perovskia atripicifolia ‘WALPPB’ in a full-sun display garden in Zeeland, Mich. with supplemental fertilizer and irrigation as needed. The new plant, ‘WALPPB’, has not been observed under all possible environments. The phenotype may vary slightly with different environmental conditions, such as temperature, light, fertility, moisture and maturity levels, but without any change in the genotype. The color descriptions are in accordance with the 2001 R.H.S. Colour Chart of The Royal Horticultural Society, London, England, except where common dictionary color terms are used.
Plant habit: erect and compact shape; oppositely branched; deciduous, semi-woody sub-shrub usually dying to the ground in most winters and re-sprouting with new stems from the base each spring;
Plant size: reaches a maximum of about 70 cm in height with an average stem height of 58 cm, and 50 to 55 cm in width; one-year old plant about 42 to 46 cm tall and 45 cm across; Root description; fibrous;
Propagation: stem cuttings; root initiation occurs in 14 to 21 days at a temperature of 20° C.;
Growth rate: moderately vigorous;
Stem description: quadangular; average 58 cm long and 4.0 nun across near base; lanulose; with herbal fragrance;
Stem color: nearest RHS 189D in upper or distal region closer to flowers and between RHS 191B and RHS 191C in lower or proximal region; nodal region same color as surrounding stem;
Internode length: average 2.0 cm
Foliage description: finely pubescent on abaxial and adaxial surfaces; compound, finely dissected; opposite lanceolate in outline, acute apex and cuneate base; about 6.5 cm long and 3.5 cm wide; with herbal fragrance;
Leaflets: further dissected; attached randomly with some alternate and some opposite; longest generally proximally; about 2.5 cm long with petioles about 1.5 mm wide; attached at about 45° angle to base; secondary leaflets about 2.0 mm long and 1.0 mm wide; some leaflets without secondary leaflets;
Leaf color: abaxial surface between RHS 138A and RHS 138B; adaxial surface nearest RHS N138B;
Leaf venation: pinnate, matching with leaf segments;
Vein color: adaxial nearest RHS N138A, abaxial lighter than RHS 192D in main vein and nearest RHS 138A in secondary veins;
Petiole: flattened abaxial to adaxial, concavo-convex; about 13 mm long and 2.0 mm across at widest point just before leaflets; color nearest RHS N138A on adaxial and lighter than RHS 192D in center and nearest RHS 138A in perimeter;
Inflorescence: main stems vertical, branched verticillate panicle; about 35 cm tall and 19 cm wide; branches in the distal ½ to ⅔ of main stem with some secondary branching in lower branches; branches at angle of 45° or less from vertical main stem;
Flower buds about one day prior to opening: about 8.0 mm long and 3.5 mm wide; ovoid; calyx with dense long pubescence about 1.0 mm long;
Bud color: calyx hairs between RHS 91B and RHS 91C; calyx nearest RHS 138B with veins of nearest RHS 77A; extended petal portion nearest RHS 96D;
Flowers: bilabiate with upper and lower lip, zygomorphic; about 12.5 mm long and 6.5 mm wide; lips fused into tube in the proximal 8.5 mm, face opening to nearly flat; up to 15 flowers per verticil and over 600 per main stem; slight fragrance herbal;
Corolla: glabrous; bilabitate; upper lip four-lobed, lower lip entire; opening to nearly a flat face; lip and lobe apices rounded; base fused into tube; tube about 8.5 mm in length and 2.0 to 2.5 mm in width; upper lobes about 1.5 mm across; lower lip about 3.5 mm across;
Corolla color: adaxial distal portion between RHS 93A and RHS 93B with proximal fused portion lighter than RHS 97D and mid-portion showing veining of nearest RHS 93B; abaxial distal portion nearest RHS 93C with the basal one third inside corolla lighter than RHS 97D;
Gynoecium: one pistil;
   Style.—about 14.0 cm long and less than 0.5 mm wide; color nearest RHS 85 C distally and lightening to lighter than RHS 85D.
   Stigma.—splitting into two and curling around in the terminal 1.0 mm; color lighter than RHS 85D.
Androecium: four, two fertile and two sterile; outer pair sterile and inner pair fertile; stamens adnate to corolla;
   Filaments (fertile pair).—about 1 cm in length and less than 0.5 mm in width; color nearest RHS 155D.
   Filaments (sterile pair).—without filament.
   Anther.—fertile pair globose, about 1.0 mm diameter, versatile; color nearest RHS 165D; sterile pair ovoid, about 0.5 mm long; color nearest RHS 155D.
Pollen: sparse in quantity; color lighter than RHS 155D;
Calyx: tubular campanulate; about 6.0 mm long and 2.5 mm across; made up of five sepals fused at base in the proximal 4.5 mm with apical 1.5 mm separate; entire, apex acute; outside densely pubescent and glandular at surface, inside glabrous; persistent for weeks after flower abscission and drop;
Calyx color: outside underneath color at flowering nearest RHS 83A and hairs between RHS 85B or RHS 85C, inside color is nearest RHS 138B distally and lighter than RHS 138D proximally with purple veins of nearest RHS 86A;
Seed: ovoid to spatulate in outline; about 2.0 mm long and 1.0 mm diameter; color nearest RHS 200D; Hardiness to at least U.S.D.A. zones 4 to 9;
   Pest and diseases resistance.—no susceptibility or resistance to diseases or pests has been observed except that which is common to Perovskia.

I claim:

1. A new and distinct cultivar of hardy Russian Sage plant, Perovskia ‘WALPPB’, as herein described and illustrated, with erect and compact habit, long flowering season, lavender blue flowers and silvery green finely dissected foliage suitable as a potted plant, as fresh or dried arrangements, for the garden as an accent or en mass.
